(19) United States
(12) Patent Application Publication
(10) Pub. No.: US 2024/0306974 A1
(43) Pub. Date: Sep. 19, 2024

(54) SYNTHETIC ECHO FROM ECG

(71) Applicant: WEST VIRGINIA UNIVERSITY BOARD OF GOVERNORS ON BEHALF OF WEST VIRGINIA UNIVERSITY, Morgantown, WV (US)

(72) Inventors: Partho SENGUPTA, Morgantown, WV (US); Naveena YANAMALA, Morgantown, WV (US)

(21) Appl. No.: 18/571,127

(22) PCT Filed: Jun. 16, 2022
(86) PCT No.: PCT/US2022/033737

§ 371 Date: Dec. 15, 2023

Related U.S. Application Data
(60) Provisional application No. 63/212,228, filed on Jun. 18, 2021.

(51) Int. Cl. A61B 5/341; A61B 5/00; A61B 5/256

(52) U.S. Cl. CPC A61B 5/341; A61B 5/0006; A61B 5/256; A61B 5/6804; A61B 5/7264; A61B 2562/222

(57) ABSTRACT

Various examples are provided related to synthetic echocardiography. In one example, a method includes receiving surface electrocardiography (ECG) signals obtained from a patient; synthesizing, through a machine learning model, a 3D model of a heart based upon the surface ECG signals; and generating a rendering of the heart based upon the synthesized model of the heart. In another example, a system includes a wearable monitoring device that can collect and transmit surface ECG signals; and a computing device that can receive the surface ECG signals obtained from a patient using the wearable monitoring device; synthesize, through a machine learning model, a 3D model of a heart based upon the surface ECG signals; and generate a rendering of the heart based upon the synthesized model of the heart. The rendering of the heart can be displayed locally (e.g., by the computing device) or transmitted to a user device for display.

### CROSS REFERENCE TO RELATED APPLICATIONS

This application claims priority to, and the benefit of, co-pending U.S. provisional application entitled “Synthetic ECHO from ECG” having Ser. No. 63/212,228, filed Jun. 18, 2021, which is hereby incorporated by reference in its entirety.

### BACKGROUND

Echocardiography is now established as a cardinal non-invasive approach for evaluation of a cardiac patient. This transformative modality has received intense scrutiny for the potential of overuse and the associated cost ramifications. In the context of telemedicine, echocardiography becomes a challenge since the devices are not easily portable and even when portable devices are available, they still need to be applied by trained technicians. Even though hand-held echocardiography has been advocated as an adjunct to physical assessment, echocardiographic evaluation cannot be undertaken remotely. Therefore, some contact between the patient and the healthcare provider is needed with either the patient making a visit to the health facility, or the technician making a field visit, defeating the purpose of telemedicine and social distancing.

### SUMMARY

Aspects of the present disclosure are related to synthetic echocardiography. In one aspect, among others, a method for synthetic echocardiography comprises receiving surface electrocardiography (ECG) signals obtained from a patient; synthesizing, through a machine learning model, a 3D model of a heart based upon the surface ECG signals; and generating a rendering of the heart based upon the synthesized model of the heart. In one or more aspects, the machine learning model can comprise a generative adversarial network (GAN) model that synthesizes ECG frames based upon the surface ECG signals. The machine learning model can comprise a frame discriminator and a sequence discriminator configured to generate a reconstruction of the heart based upon the synthesized ECG frames and ground truth frames. The frame discriminator and sequence discriminator can produce a cohesive video of the heart that exhibits natural cardiac movements. The rendering can comprise the cohesive video.

In various aspects, the surface ECG signals can be collected and transmitted by a mHealth device worn by the patient. The surface ECG signals can comprise 12-lead ECG signals obtained from the patient in real time. The surface ECG signals can be received by a computing device from the mHealth device through a communications network. The computing device can be a backend server. In some aspects, the method can further comprise transmitting the rendering of the heart to a user device for display. The user device can be a virtual reality/augmented reality (VR/AR). The rendering of the heart can comprise a cohesive video of the heart. The rendering of the heart can be transmitted from a backend server.

In another aspect, a system for synthetic echocardiography comprises a wearable monitoring device configured to collect and transmit surface electrocardiography (ECG) signals; and a computing device comprising processing circuitry configured to: receive the surface ECG signals obtained from a patient using the wearable monitoring device; synthesize, through a machine learning model, a 3D model of a heart based upon the surface ECG signals; and generate a rendering of the heart based upon the synthesized model of the heart. In one or more aspects, the machine learning model can comprise a generative adversarial network (GAN) model that synthesizes ECG frames based upon the surface ECG signals. The machine learning model can comprise a frame discriminator and a sequence discriminator configured to generate a reconstruction of the heart based upon the synthesized ECG frames and ground truth frames. The rendering can comprise a cohesive video of the heart produced by the frame discriminator and sequence discriminator. In various aspects, the surface ECG signals can comprise 12-lead ECG signals obtained from the patient in real time. The computing device can be a backend server. The computing device can be configured to transmit the rendering of the heart to a user device for display.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A and 1B illustrate an example of machine learning-based models enhanced with computational models that may be used for telemedicine assessments, in accordance with various embodiments of the present disclosure.

FIG. 2 illustrates an example of an approach to establish accurate reconstruction of a heart from electrocardiography (ECG) signals, in accordance with various embodiments of the present disclosure.

FIG. 3 illustrates an example of development of an end-to-end echo model of the heart for ECG-driven animation, in accordance with various embodiments of the present disclosure.

FIG. 4 illustrates examples of innovative components of the synthetic echocardiography, in accordance with various embodiments of the present disclosure.

FIG. 5 illustrates an example of a system that can be used for synthetic echocardiography, in accordance with various embodiments of the present disclosure.

### DETAILED DESCRIPTION

Disclosed herein are various examples related to synthetic echocardiography. Using a non-invasive and easy to use method such as ECG, without direct patient-provider contact, and converting that information into a simulated echocardiographic output can, if accurate, have a transformative impact on the practice of telemedicine. Combining the simplicity and information content of the ECG with the power and creativity of artificial intelligence offers a solution. Reference will now be made in detail to the description of the embodiments as illustrated in the drawings, wherein like reference numbers indicate like parts throughout the several views.

Deep learning models developed based on global electrocardiography (ECG) measures can be effective in capturing parameters associated with cardiac structure and function and such information can be useful in reconstructing a simulated model of an interactive real-time beating heart. An AI based system can be developed that uses ECG recordings (which can be easily conducted at the patient's location without expert technical assistance) as the input to generate and/or render an echocardiographic reconstruction for display of that patient's beating heart in real-time. Such a reconstructed echocardiographic rendering can prove to be an important assistive tool to cardiologists offering their expertise remotely.

Mechanics of the heart begin with electrical impulses and continuous electromechanical and mechano-electric feedbacks that modulate the cardiac muscle performance. How exactly, the intricate electrical and mechanical relationships translate into global performance of a beating heart is important to understand. Surface ECG measures and maps the electrical activity of heart while echocardiography non-invasively slices through the heart to get snapshots of the structure and function of heart. Connecting these two modalities of heart appraisal into a single domain presents difficulties. In this regard, what the human mind grapples to comprehend can be explicitly modeled using the principles of artificial intelligence. The proposed methodology utilizes an artificial intelligence approach (using, e.g., generative adversarial networks) to exploit the interpretations offered by computational modeling to reconstruct the beating heart structure and function by recording the surface ECG. The computational models generated from the synthetic and native echocardiogram can serve as ‘digital twins’ for dynamic comparison of cardiac structure and function in healthy and diseased populations.

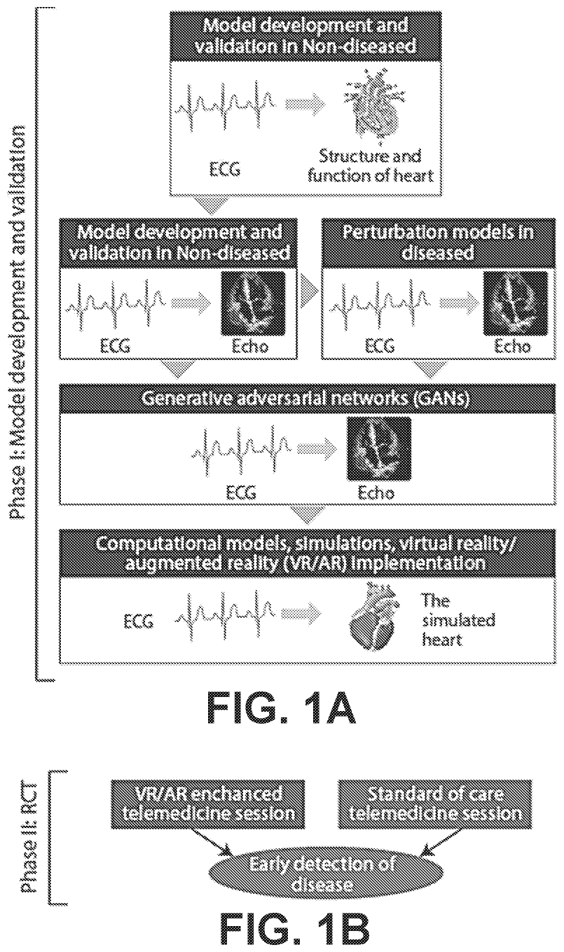

A novel, transformative, and assistive digital-health technology is disclosed that can boost access to affordable, high-quality, sophisticated telehealth consultative services. As illustrated in FIG. 1A, machine learning-based models enhanced with computational models derived from a rich repository of existing datasets on ECG and the structure and function of heart assessed via echocardiography can be developed and validated. Initially, modeling of the structure and function of the heart can be developed under non-diseased conditions. These models can be extended for echocardiography under non-diseased and diseased condition. Machine learning including, e.g., generative adversarial networks (GANs), can be used for the development of simulations that can be implemented in, e.g., virtual reality/augmented reality (VR/AR) display.

A remotely measured electrocardiographic (ECG) input can be used and translated into a live, simulated, and generated set of images that mimic echocardiography and can be presented with virtual reality/augmented reality. The virtual cardiac model can be developed as a surrogate of cardiac physical assessment entirely from ECG that is recorded remotely and transmitted live to the caregiver during a telemedicine visit. An artificial intelligence (AI)-based conversion can transform the raw ECG waveform into the echocardiographic output. This may be based on powerful machine-learning platforms like generative adversarial networks (GAN). The generated data can be represented in a physician-friendly manner using, e.g., virtual reality (VR) or other appropriate rendering methodologies. In this way, an accurate AI-based solution can be implemented to reconstruct echocardiographic data and images from remotely measured ECG.

As illustrated in FIG. 1B, the disclosed methodology may be used for tele-evaluation to enhance remote assessments in a specialty clinic by a cardiologist such as, e.g., triaging for in-person visits and advanced assessment. Telemedicine sessions can use a virtual reality/augmented reality-driven solution enhanced through the power and accuracy of AI. A randomized control trial (RCT) can be conducted to Demonstration of the clinical utility of the proposed approach can be validated through, e.g., a randomized control trial (RCT). Using ECG→Echocardiography as a template, this approach can use the information on non-invasive assessments like instantaneous blood pressure, respiration, and body temperature along with ECG and combine all the information to generate echocardiographic reconstruction, computerized tomographic images from simple clinical data.

Several data-driven and/or machine learning techniques including, e.g., deep neural networks, long short-term memory networks (LSTM), and generative adversarial networks (GAN) can be utilized for the automatic synthesis and animation based upon a signal. For example, a realistic 3D facial animation model called voice-operated character animation (VOCA) takes a speech signal as input and realistically animates a wide variety of faces including different speaking styles. Thus, data-driven approaches can provide a baseline measure that one can compare the synthesized motions through evaluation of how much they deviate from the original data.

Similarly, physiological and biological signals such as heart rate, ECG, EMG, temperature, and others have been exploited for emotion detection, medical, and biometrics purposes. The ECG signals can be used as a stamp or biomarker to reconstruct echocardiographic images or videos. ECG signals may be used to learn rules/relationships specific to Echo (e.g., EF, strain, LV mass, dimensions, etc.). Generative models may be used in the synthesis of ECG-driven echocardiographic animations. Given an input ECG signal, with the base template images of end-systolic and end-diastolic frames, an end-to-end echo model of the heart can be generated or synthesized using temporal GANs.

Learning vocabulary associated with cardiac structure and function can be defined by echocardiographic studies and its relation to time-resolved 12-lead ECG. The extracted features, relationships, and properties of ECG signals over a given length (e.g., a single heartbeat) can be used to map a set of cardiac poses (e.g., end-systolic, end-diastolic, etc.) from echocardiographic videos—visual counterparts of cardiac structure/function—during model training. This can be done by considering the position and the velocity of different cardiac features and learning a probability distribution over the different configurations of motions in a cardiac cycle or by representing them as a set of vectors. Animations can then be synthesized by performing suitable operations within the interpolation space of the learned models. These approaches thus may even allow for representing cardiac motions in the form of abstract functions rather than as a complex biomechanical system.

### Machine Learning Approaches for Predicting Cardiac Structure and Function from ECGs

The ECG remains the most widely used diagnostic test for the characterization of cardiac structure and electrical activity. Machine-learning techniques can be employed to extract meaningful features from surface ECGs for the efficient risk stratification of patients at risk for heart failure. For example, machine-learning can enable the classification of asymptomatic LV systolic dysfunction or detection of abnormal myocardial relaxation with ECGs. Moreover, it has been demonstrated that a machine-learning-based approach enabled quantitative estimation of LV diastolic dysfunction using continuous regression values of LV relaxation velocities. A combination of convolutional neural networks and hidden Markov models can be employed to build a patient-level ECG profile. The ECG profile can comprise a feature vector that can be used to predict features of cardiac structure (left ventricular mass, left atrial volume, and mitral annulus e-prime) and disease (pulmonary arterial hypertension, hypertrophic cardiomyopathy, cardiac amyloid, and mitral valve prolapse). Models trained using patient-level ECG profiles can enable quantitative estimates of left ventricular mass and mitral annulus early diastolic relaxation velocity with good discrimination for left ventricular hypertrophy and diastolic dysfunction as binary traits. This may be used to derive features of cardiac structure (LV volumes, mass, geometry) and function (EF, relaxation velocities, strain) that can be utilized as input for developing a 3D model of a beating heart.

### Computational Modeling of Cardiac Structure and Function

Mechanistic, highly complex multi-scale physiological models of the heart have been developed for many years in academia and are now being deployed by industry. These computational models can be used to solve the governing equations underlying complex systems and simulate their behavior. Computational cardiac models can simulate the electrophysiology and/or mechanical deformation of cardiac myocytes, tissue, or even the whole heart. The predictive capability of such computational models based on the fundamental laws of physics can enable the routine use of the models in a multitude of engineering applications. With increasing model fidelity under tight validation scrutiny, the modeling of cardiac structure and function can provide accurate simulations.

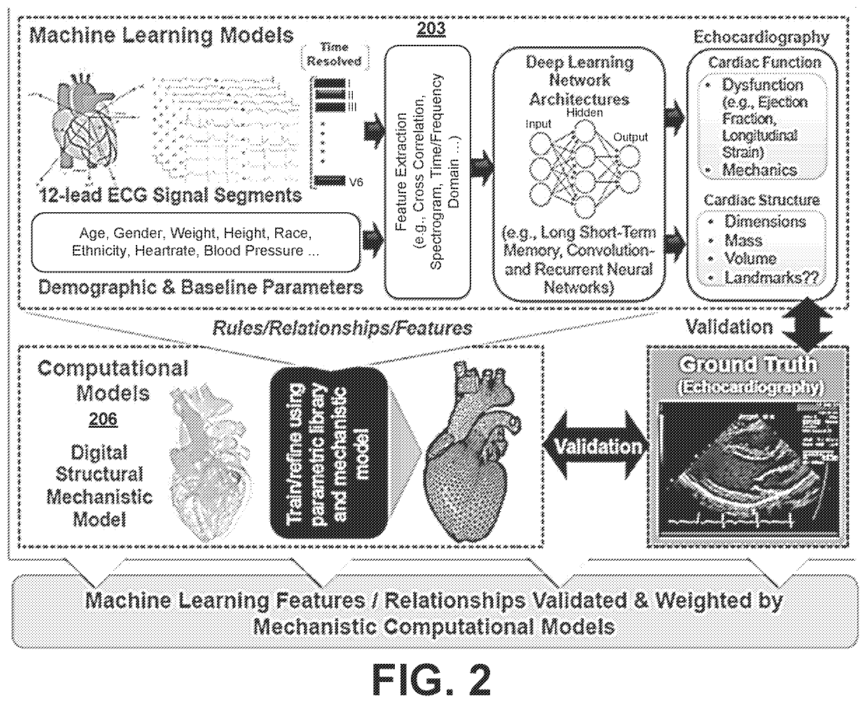

A novel AI-based solution can be developed that has the potential to revolutionize the practice of telemedicine for cardiac consultations. Deep learning models developed based on global electrocardiography (ECG) measures may be effective in capturing parameters associated with cardiac structure and function and that such information can be useful in reconstructing a simulated model of an interactive real-time beating heart. An accurate interface between the ECG and the heart structure and function can be achieved in two phases, which are illustrated in FIG. 2. As shown in 203, raw and transformed features of the ECG signal can be used for developing predictive models of cardiac mechanical function. Time-resolved deep learning models can be developed for predicting the dynamic structural/functional features of a beating heart from, e.g., the standard 12 lead ECG. In 206, parametric mechanistic models of heart can be developed that can be further used to regenerate the measured ECG and validate the predictive models.

Synthesis of echocardiographic videos from surface ECG. Machine learning models can be developed and validated that establish a clinically meaningful relationship between ECG. Advanced AI and computational methods can be used to derive clinical variables from the surface ECG that define the structure and function of a beating heart. FIG. 2 illustrates an example of an approach to establish accurate reconstruction of the structure and function of the heart from a 12-lead ECG signal. This can be achieved through model development, model evaluation and model validation.

Model Development: ECG-driven deep-learning based models can be developed that can accurately predict the echocardiographic parameters. Deep-learning based models can be developed that can accurately predict the echocardiographic parameters—those describing function and structure—from surface ECG. Features representing ECG and QRS spectrum/signals can be computed or extracted, e.g., in a time-scale domain by using fast Fourier transforms (FFT), short-time Fourier transform (STFT) and/or continuous wavelet transform (CWT) as appropriate. The transformed ECG feature dataset can also include features from distance distribution matrices based on entropy calculations, gray-level co-occurrence matrices, or combined features, such as, e.g., morphologies, RR intervals and beat-to-beat correlations. Such high dimensional signal processed and raw ECG features can be combined with demographic/clinical features of patients to develop deep learning-based predictive models of cardiac structure/function.

Predictive models of cardiac structure and function can be developed using, e.g., (a) 1D/2D convolutional neural networks (CNN) depending on the type of data being employed; (b) recurrent neural networks (RNN), and long short-term memory (LSTM) network as a logical choice for capturing both temporal dependencies and handling inputs of various lengths; or (c) a combination of both CNN and RNN, often called CRNN, for handling long ECG signals with varying sequence lengths and multi-channel inputs simultaneously. While CNN can used to extract local features from an ECG sequence, an RNN can summarize local features along the time dimension to generate global features. The developed models may be useful in deriving features/rules/relationships of cardiac structure (LV volumes, mass, geometry) and function (EF, relaxation velocities, strain) which can be utilized as an input for a 3D model of a beating heart.

Model Evaluation: The parametric relationships derived from predictive models of ECG can be reevaluated within a cardiac computational model. To correlate measured ECG data on the chest to 3D simulated ECG on the heart surface, a heart model such as, e.g., the Dassault/Simulia Living Heart Human Model (LHHM) can be used. The LHHM is a dynamic high-fidelity model of a normal (healthy), 4-chamber adult male human heart. It includes well-defined anatomic details of the heart as well as proximal vasculatures, such as the aortic arch, pulmonary artery, and superior vena cava. The LHHM is a complex, multi-physics model, where each of the multi-physics elements can be altered. For example, the electrical & electromechanical coupling part of LHHM can be parameterized to facilitate the physics-based validation exercises. Although the heart model represents a healthy heart, abnormal (diseased) states can also be examined by modifying any of its geometry, material properties, and/or loads and boundary conditions.

The dynamic response of the human cardiac models is governed by realistic electrical, structural, and fluid (blood) flow physics. As such, the electrical behavior of the heart by itself or its coupled electromechanical behavior wherein the mechanical response is driven by electrical excitation can be examined. A bottom-up spatio-temporal evolution of electric potential spreads through the ventricles of the heart. After a short period of complete depolarization, repolarization spreads gradually across the left and right ventricles and atria to bring the heart back to its unexcited baseline state. The mechanical deformation follows the electrical signal. As the electrical potential returns to its baseline state, the deformation gradually decays, the heart relaxes, and prepares for the next filling phase. The mechanical response of cardiac muscle consists of passive and active components. The active muscle stress is driven by changes in the electric potential and is a function of the contractility scaling factor. The contractility scaling factor can be used to directly scale the computed ejection fraction. To compare the simulated excitation sequences from the cardiac model with the clinical ECG data, the simulated excitations can be extrapolated to the corresponding electrode positions. The temporal evolution of the electrical potential at each electrode position can be determined based on the torso conductivity, intracellular conductivity, and the electrode position.

The electrical signal extrapolation can allow for the analysis of the recorded or measured physical ECG data from the opposite direction. That is, if the ECG is mapped back to the heart (making appropriate patient age, size, BMI, etc. allowances), then the measured ECG signal can be directly used as an input to the 3D simulated human cardiac models. Therefore, allowing possible heart responses to the same measured ECG signal to be used for ML model training. The cardiac models can use a “smart geometry” module for investigating geometric variations and geometric parameterization. This module allows mapping of ML/DL data back to a 3D Model for VR visualization of very high quality, as shown in FIG. 2. The Dassault Living Heart Human Model (LHHM) is considered to be the industry-leading solution for modeling of the complete human heart, however alternative models such as, e.g., the Siemens Healthineers (SHS) model (research code), ANSYS/LS Dyna (commercial code), and several academic codes (e.g. Oxford (CHASTE), Stanford) with similar capabilities could be applied.

Mechanistic model simulations can be developed for categorized disease cases and pathologies. The diseased heart states can be computationally studied by modifying any and all of the cardiac model's geometry, material properties, and/or loads and boundary conditions. For example, myocardial infarction can be modeled by providing stiffer material properties for the infarcted regions of the heart. The ultrasound echo-based computational modeling approach can be used to quantify the material properties of the infarcted heart by adjusting the model parameters to match echo volume data. The mechanical model can be altered based on ECG input to emulate disease cases like systolic and diastolic dysfunction. Input to the electrical cardiac models can come from measured ECG data of diseased patients and drive the mechanical model, to determine which part of the heart is affected. The electrical properties and/or mechanical properties of the heart model can be tuned to regenerate the measured ECG and validate the model. The mechanical model may also simulate how the disease affects electrical behavior.

Model Validation: The ECG-generated synthetic information of cardiac structure and function as an input to 3D computational models can be validated to confirm that they are mechanically and hemodynamically realistic. A first validation can demonstrate that the ML correlations between the electrical and mechanical model are physically realistic. An ECG signal (waveform) variation/perturbation, which can cause a shift in the ML model, can be tested to have the same trend or effect in the average patient as defined by the 3D human cardiac models. This validation can determine if the cardiac response from ML is physical (e.g., by looking at ejection fraction or strain). To achieve this, the default models including LHHM can be used as an average human heart model. For commercialization of a telemedicine product, this approach can be refined using patient-specific data. The validation can also be used to filter correlations from the ML, e.g., to remove trends or effects, which cannot be reproduced by mechanistic models. The use of the cardiac mechanistic model, similar to LHHM, can be used to understand why ML might have detected correlations. In other words, the mechanistic model can be used to a) validate the ML algorithms; b) filter correlations created by the ML algorithm; and/or c) used to select the appropriate correlations (‘prune’ the correlations) for patient diagnostic purposes.

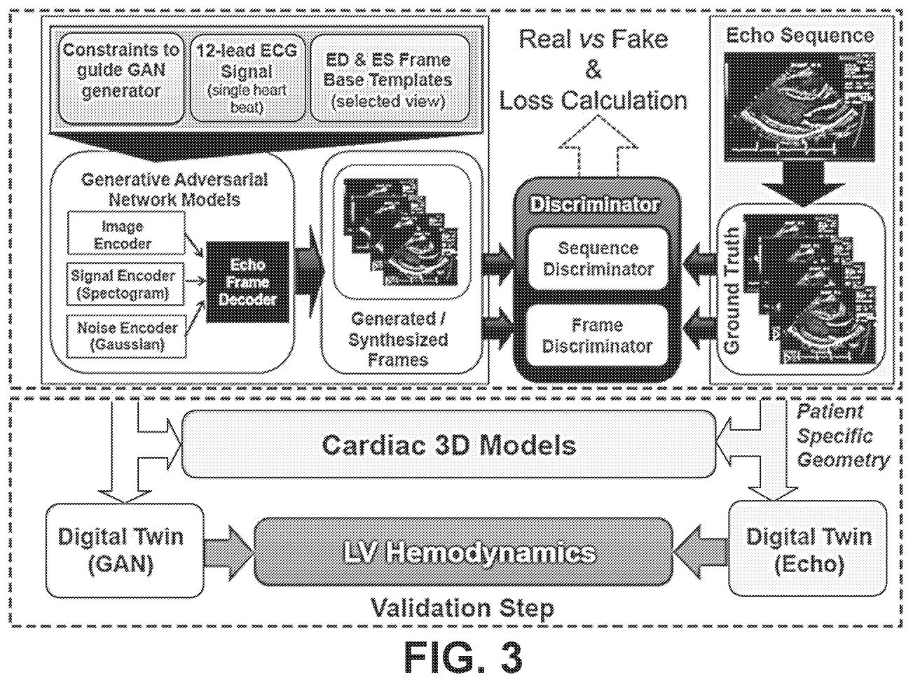

An interactive real-time beating-heart echo model can be generated from patient 12-lead ECG using the developed constraints. FIG. 3 illustrates an example of an overall approach to develop an end-to-end echo model of the heart using temporal GANs for ECG-driven animation of selected views of echocardiogram. An end-to-end echo model of the heart can be output using temporal GANs for ECG-driven animation of the parasternal long-axis and apical 4-chamber views of echocardiogram. An encoder-decoder architecture can be employed as part of the GAN to synthesize echo sequence animations from ECG. To capture different aspects of natural echocardiogram, two different discriminators (a frame discriminator and a sequence discriminator) can be used to achieve a high-quality reconstruction of the echo frames and to generate a cohesive video from synthetic frames that exhibits natural cardiac movements. The GAN-synthesized frames and the ground truth frames can be used to build the 3D geometry of the heart (cardiac 3D models) to predict hemodynamic characteristics such as chamber sizes, ejection fraction, and strain features.

Generative Adversarial Networks (GANs) can be used to model echocardiographic images from the ECG signal and constraints identified in FIG. 2. GANs represent a class of machine learning techniques that, given a training set, can generate new data with the same statistics as the training set. The GANS are a set of neural networks that compete against each other (as adversaries) in the context of a game. Thus, the improvement in gain for one model occurs at the cost of a corresponding loss in the performance of a competing network. The final model is thus the result of a ‘win’ between a generator and a discriminator that is continuously updated.

The trained ML and computational models can be used as the conditioning based on which a set of echocardiographic images at different timestamps of the ECG signal can be generated. These generated images can be compiled into a video graphic simulation that the healthcare provider can see live during a telemedicine visit. For reconstructing echocardiographic images, GANs that draw the predictive models can use three sets of inputs—the 12 lead ECG signal, the temporally coinciding end-diastolic and end-systolic echocardiographic frame sequences, and the learned physiological constraints. FIG. 3 illustrates an example of the architecture for ECG-driven echocardiographic image synthesis. As shown, a single generator and two discriminators can be used. The generator network can have an encoder-decoder structure and can be further divided into sub-networks to assume a latent representation that accounts for the image, ECG signal identity/content, and/or temporally independent spontaneous cardiac motions. The latent representation for each frame can then be constructed by concatenating the image, signal, and noise components.

Two different discriminators, a frame and sequence discriminator, can be used to capture different aspects of natural echocardiogram. While the frame discriminator can achieve a high-quality reconstruction of the echo frames, the sequence discriminator can ensure that the synthetic frames generated form a cohesive video which exhibits natural cardiac movements. In simple terms, the adversarial training with the frame discriminator can be employed to ensure that the generated frames are realistic. Further, the sequence discriminator can be employed to distinguish between real and synthetic videos. Thus, the GANs can be trained using real and fake inputs and outputs to reduce the loss function.

Difficulties can arise in training GANs. For example, three types of problems can be experienced during GAN training.
a. Non-convergence: This a common occurrence in GAN training. The Model parameters tend to oscillate and not converge to the final solution. To address this potential challenge, hyperparameter tuning (using grid search) can be conducted with regards to the loss function, the optimizer, and/or learning rates suitable for the data at hand.
b. Model collapse: In this situation, the generator collapses and fails to produce sufficient varieties of the samples. Data augmentation and hyperparameter training can be used to address this situation should it occur.
c. Diminished gradients and imbalance: GANs comprise two main components—the generator and the discriminator. If the GAN discriminator becomes too successful, then the GAN doesn't enrich its learning experience and is not efficient. If an imbalance remains between the generator and discriminator it tends to overfit and have less accuracy. GANs provide optimum performance when they reach a balance between the generator and the discriminator components (a condition called Nash equilibrium). When there remains an imbalance the GANs can over or under perform depending on the direction of imbalance. Addressing this potential challenge is addressed when it occurs and includes hyperparameter tweaking to ensure acceptable sensitivity to hyperparameters as well as the generalizability of the final model.

The original echo and GAN-synthesized echo model can be compared in their abilities to produce realistic 3D computational heart models and predict hemodynamics (e.g., left ventricular hemodynamic assessment of the patient's heart). A combination of a) GANs; b) patient-specific geometry reconstruction using ground truth echocardiography data; c) patient-specific geometry reconstruction using GAN-synthesized echocardiographic images; and d) using the digital twin hearts reconstructed in b) and c) can predict parameters of clinical interest that capture left ventricular hemodynamics. This can produce two sets of echocardiographic frames—the GAN-synthesized frames and the ground truth frames. Each of these sets can be used to model the three-dimensional geometry of the heart in steps b) and c). These reconstructions can be referred to as the digital twin (GAN) and the digital twin (Echo). These digital twins can be compared for their ability to work as an input to the computational models for predicting hemodynamic characteristics such as left ventricular flow, filling pressures, regional and global strain characteristics, stroke volume, etc. Using such standard hemodynamic criteria used in clinical practice, the accuracy and incremental value of the models can be benchmarked for characterizing cardiac health and disease.

Comparison of conventional patient encounters with telepresence. Conventional patient encounters can be compared with holographic telepresence using extended reality-based clinical exams for diagnostic patient triage or clinical decision making in real-time. A randomized control trial (RCT) utilizing the Al-based solution as an assistive technology in a telemedicine clinic can be conducted to determine the improvement in referral decision making can be used. A participating physician can present a cardiac patient that they are referring to a cardiologist using a conventional telemedicine visit protocol to discuss a decision about the need for an in-person visit and an advanced evaluation. During this virtual visit, the physician and remote cardiologist can interact with the reconstructed echocardiographic information and be provided with an informed decision to change the originally taken referral decision. The influence of the Al-based reconstructive solution on decision-making in real-time patient encounters can subsequently be evaluated to determine the utility of VR/AR solution by comparing the standard of care vs VR/AR implemented care.

A geometric/mechanistic model can be integrated into a VR/AR environment using parametric anatomy models. Virtual Reality can provide a 3D beating heart, which can be moved, rotated, examined from all angles, and taken apart to see individual components and how they interact. The 3D displays can be useful both for enhanced understanding by a clinician and for communicating with the patient and other individuals.

Implementation: Software tools can automatically generate VR displays from the standard output of commercial Finite Element and computational fluid dynamics simulation software. A range of VR devices is currently supported, including, e.g., zSpace, HTC Vive Pro, and Oculus Quest. Animations of the VR can also be displayed on conventional computer monitors, although the 3D immersed effects will be lost. Field quantities such as electrical potential, pressure, or strain vs time can be displayed, and the parts can be separated for inspection. For example, by fitting a parametric model such as the “smart geometry” feature of the LHHM to the ML generated 2D echocardiograms, a 3D VR of the beating heart can be produced.

During the telemedicine visit, the patient can attach a wearable device that collects and transmits the ECG signals. For example, wearable jackets can be configured to collect and transmit ECG signals remotely through, e.g., a communications network (e.g., the Internet, a cellular network, WAN, LAN, or other appropriate communications network). In some cases, the ECG information can be transmitted to a remotely located computing device via a local user device (e.g., computer, tablet, smartphone, etc.) in communication with the ECG device. Miniaturized mHealth devices can also record and transmit 12-lead ECG signals. In a telemedicine scenario, the ECG device can collect and transmit the ECG information to, e.g., a backend server where a synthetic echo application can generate the VR/AR solution. The server can execute the application and communicate with a user device (e.g., VR/AR system, computer, tablet, smartphone, or other appropriate computing or display device) at the physician's office to display the VR/AR solution as, e.g., a GAN-synthesized echocardiographic video and a reconstructed digital twin heart alongside the display of the original ECG signal. In addition to telemedicine with the patient, the physician may conduct a telemedicine session to present the case to a remote cardiologist using the VR/AR solution. Both may view the echocardiographic video simulation to discuss the patient's scenario. The VR/AR solution can enhance the ability to detect, e.g., left ventricular dysfunction, heart failure with reduced or preserved ejection fraction.

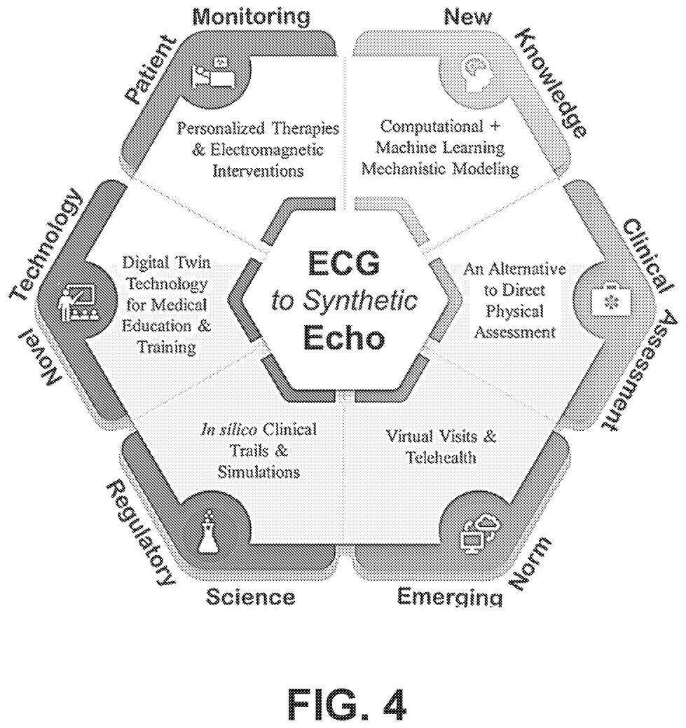

Referring now to FIG. 4, shown are examples of innovative components of the synthetic echocardiography technology.
New knowledge through the marriage between computational modeling and machine learning: Traditional computational modeling entails a model that is conceptualized first, supported by assumptions, enhanced by available data, and then tested for accuracy. Machine learning, on the other hand, attempts to fit data to standard mining architectures and is, therefore, primarily data driven. The disclosed approach unifies these modeling approaches to arrive at a stable and robust prediction of the structure and function of the heart from the recording of the heart's electrical activity. This can ensure accurate translation of the heart's electrical and mechanical activity.
The link between electrical activity and heart structure and function for patient monitoring: There exists no such data or an attempt to link the record of the electrical activity of the heart with its structure and function in a generative setting. A retrospective or reverse-directed assessment of echocardiographic evaluation can go back to the ECG. The electrical activity of the heart can be treated as the driver of the echocardiographic manifestation of the underlying physiology and pathology.
Novel technology using a combination of GANs with VR/AR to generate echocardiographic simulation in real time: The AI-based synthetic echo methodology can enrich the physician's experience of informed decision making.
In silico clinical trial opportunities for regulatory science: An in silico clinical trial is an individualized computer simulation that can be used in the development or regulatory evaluation of a medicinal product, device, or intervention. The FDA is already planning for a future in which more than half of all clinical trial data will come from computer simulations and in silico clinical trials. For example, the proposed model can become an effective biomarker to examine the effects of drugs on body-surface ECG parameters using realistic 3D models of the ventricles derived from surface ECG. The individualized predictions could also be used for therapy planning and more realistic shared decision-making.
Telemedicine as the emerging norm: In the era of an ongoing global pandemic, telemedicine has already become a cornerstone of healthcare delivery in the developed and developing world alike. While the benefits of telemedicine are evident, its limitations—mainly the inability to conduct investigations (including laboratory investigations)—can undermine the use of telemedicine. The disclosed approach can be highly effective at addressing the timely demand for telemedicine.
Independent cardiovascular system clinical assessment: Cardiovascular physical examination has changed little since the 19th century, but medical practice, in the meantime, has changed substantially. The assessment of the cardiovascular system can be accomplished using surrogate measures obtained directly from the synthetic echocardiograms derived from surface ECG. This can reduce the need for costly scanning and quality of imaging for deriving information regarding cardiac structure and function.

Cardiac simulation using computational modeling is traditionally rooted in natural sciences and engineering and typically includes mathematical models that have a long history in basic science applications and device design. Machine learning and AI-based approaches can bridge the existing gap between data-based and knowledge-based cardiac modeling. Diagnosis and surveillance, patient selection, therapy optimization, or personalization can be enabled the use of ‘a digital twin’ constructed using only ECG data. The framework for recovering cardiac structure and function simply from surface ECG has the potential to transform the entire field of cardiology since ECG is a cost-effective and mainstay technique in cardiac diagnostics in the clinical or hospital setting. The future of cardiac assessment is shifting towards inexpensive wearable or smartphone-based ECG monitors that represents a novel method for patient engagement in cardiology. Patient clinical examination and decisions during virtual or in-person encounters can be made using a patient-specific beating heart model that is recovered and overlaid in real-time just using surface ECG. A personalized mechanistic model of beating heart constructed from the surface ECG can provide an opportunity for in silico representation of cardiac structure and function incorporating electromechanical and structural remodeling in cardiac disease as the first line of screening for new therapies and approaches, including pharmacological intervention.

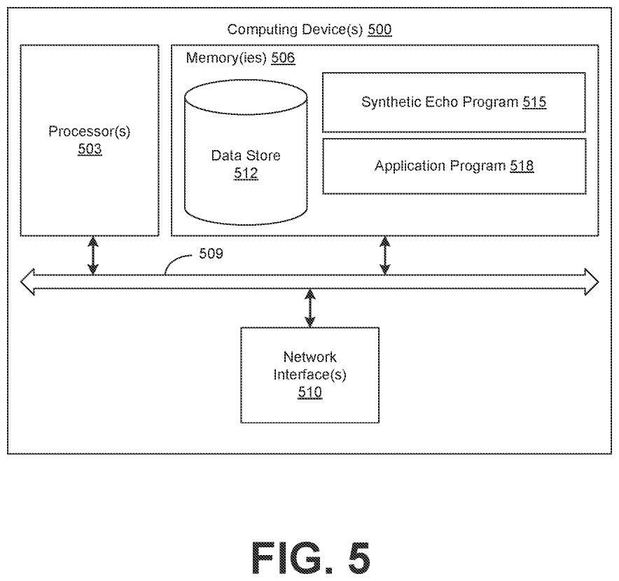

With reference to FIG. 5, shown is a schematic block diagram of a computing device 500 that can be utilized to enhance pocket US and evaluate risk stratification using the described techniques. In some embodiments, among others, the computing device 500 may represent a mobile device (e.g. a smartphone, tablet, computer, etc.). Each computing device 500 includes at least one processor circuit, for example, having a processor 503 and a memory 506, both of which are coupled to a local interface 509. To this end, each computing device 500 may comprise, for example, at least one server computer or like device. The local interface 509 may comprise, for example, a data bus with an accompanying address/control bus or other bus structure as can be appreciated.

In some embodiments, the computing device 500 can include one or more network interfaces 510. The network interface 510 may comprise, for example, a wireless transmitter, a wireless transceiver, and a wireless receiver. As discussed above, the network interface 510 can communicate to a remote computing device using a Bluetooth protocol. As one skilled in the art can appreciate, other wireless protocols may be used in the various embodiments of the present disclosure.

Stored in the memory 506 are both data and several components that are executable by the processor 503. In particular, stored in the memory 506 and executable by the processor 503 are a synthetic echo program 515, application program 518, and potentially other applications. Also stored in the memory 506 may be a data store 512 and other data. In addition, an operating system may be stored in the memory 506 and executable by the processor 503.

It is understood that there may be other applications that are stored in the memory 506 and are executable by the processor 503 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 506 and are executable by the processor 503. In this respect, the term “executable” means a program file that is in a form that can ultimately be run by the processor 503. Examples of executable programs may be, for example, a compiled program that can be translated into machine code in a format that can be loaded into a random access portion of the memory 506 and run by the processor 503, source code that may be expressed in proper format such as object code that is capable of being loaded into a random access portion of the memory 506 and executed by the processor 503, or source code that may be interpreted by another executable program to generate instructions in a random access portion of the memory 506 to be executed by the processor 503, etc. An executable program may be stored in any portion or component of the memory 506 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 506 is defined herein as including both volatile and nonvolatile memory and data storage components. Volatile components are those that do not retain data values upon loss of power. Nonvolatile components are those that retain data upon a loss of power. Thus, the memory 506 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 503 may represent multiple processors 503 and/or multiple processor cores and the memory 506 may represent multiple memories 506 that operate in parallel processing circuits, respectively. In such a case, the local interface 509 may be an appropriate network that facilitates communication between any two of the multiple processors 503, between any processor 503 and any of the memories 506, or between any two of the memories 506, etc. The local interface 509 may comprise additional systems designed to coordinate this communication, including, for example, performing load balancing. The processor 503 may be of electrical or of some other available construction.

Although the synthetic echo program 515 and the application program 518, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the synthetic echo program 515 and the application program 518, that comprises software or code can be embodied in any non-transitory computer-readable medium for use by or in connection with an instruction execution system such as, for example, a processor 503 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the synthetic echo program 515 and the application program 518, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 500, or in multiple computing devices in the same computing environment. Additionally, it is understood that terms such as “application,” “service,” “system,” “engine,” “module,” and so on may be interchangeable and are not intended to be limiting.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A method for synthetic echocardiography, comprising:
receiving surface electrocardiography (ECG) signals obtained from a patient;
synthesizing, through a machine learning model, a 3D model of a heart based upon the surface ECG signals; and
generating a rendering of the heart based upon the synthesized model of the heart.

2. The method of claim 1, wherein the machine learning model comprises a generative adversarial network (GAN) model that synthesizes ECG frames based upon the surface ECG signals.

3. The method of claim 2, wherein the machine learning model comprises a frame discriminator and a sequence discriminator configured to generate a reconstruction of the heart based upon the synthesized ECG frames and ground truth frames.

4. The method of claim 3, wherein the frame discriminator and sequence discriminator produce a cohesive video of the heart that exhibits natural cardiac movements.

5. The method of claim 4, wherein the rendering comprises the cohesive video.

6. The method of any of claims 1-5, wherein the surface ECG signals are collected and transmitted by a mHealth device worn by the patient.

7. The method of claim 6, wherein the surface ECG signals comprise 12-lead ECG signals obtained from the patient in real time.

8. The method of claim 6, wherein the surface ECG signals are received by a computing device from the mHealth device through a communications network.

9. The method of claim 8, wherein the computing device is a backend server.

10. The method of any of claims 1-9, further comprising transmitting the rendering of the heart to a user device for display.

11. The method of claim 10, wherein the user device is a virtual reality/augmented reality (VR/AR).

12. The method of claim 10, wherein the rendering of the heart comprises a cohesive video of the heart.

13. The method of claim 10, wherein the rendering of the heart is transmitted from a backend server.

14. A system for synthetic echocardiography, comprising:
a wearable monitoring device configured to collect and transmit surface electrocardiography (ECG) signals; and
a computing device comprising processing circuitry configured to:
receive the surface ECG signals obtained from a patient using the wearable monitoring device;
synthesize, through a machine learning model, a 3D model of a heart based upon the surface ECG signals; and
generate a rendering of the heart based upon the synthesized model of the heart.

15. The system of claim 14, wherein the machine learning model comprises a generative adversarial network (GAN) model that synthesizes ECG frames based upon the surface ECG signals.

16. The system of claim 15, wherein the machine learning model comprises a frame discriminator and a sequence discriminator configured to generate a reconstruction of the heart based upon the synthesized ECG frames and ground truth frames.

17. The system of claim 16, wherein the rendering comprises a cohesive video of the heart produced by the frame discriminator and sequence discriminator.

18. The system of any of claims 14-17, wherein the surface ECG signals comprise 12-lead ECG signals obtained from the patient in real time.

19. The system of any of claims 14-18, wherein the computing device is a backend server.

20. The system of claim 19, wherein the computing device is further configured to transmit the rendering of the heart to a user device for display.
